(19) United States
(12) Patent Application Publication
(10) Pub. No.: US 2007/0233149 A1
(43) Pub. Date: Oct. 4, 2007

(54) Injection Device, Especially for Bone Cement

(75) Inventors: Marc Bohner, Grenchen (CH); Paul Heini, Wabern (CH)

Correspondence Address: RANKIN, HILL, PORTER & CLARK LLP, 38210 Glenn Avenue, WILLOUGHBY, OH, 44094-7808, US

(73) Assignee: DR. H. C. ROBERT MATHYS STIFTUNG, Bettlach (CH)

(21) Appl. No.: 10/597,502

(22) PCT Filed: Feb. 6, 2004
(86) PCT No.: PCT/CH04/00067

§ 371 Date: Jul. 27, 2006

(51) Int. Cl. A61B 17/58

(52) U.S. Cl. 606/93

(57) ABSTRACT

The invention relates to an injection device (1), especially for bone cement, comprising A) a syringe base (3) having a longitudinal axis (2), a front end (6), a connecting piece (8) disposed on the front end (6) and having a coaxial bore (21), and a coaxial cavity (4); B) an injection plunger (5), coaxially displaceable in the cavity (4); and C) a cannula (13) which can be linked with the connecting piece (8) and which has a center bore (14) and a rear end (15). The injection device is characterized in that D) the syringe base (3), on its front end (6), has a transition segment (22) having a bore (9) linking the cavity (4) with the bore (21) in the connecting piece (8), E) the bore (9) in the transition segment (22) and the center bore (14), at least on the rear end (15) of the cannula (13), having the same orthogonal cross-sectional area q relative to the longitudinal axis (2).

### BACKGROUND OF THE INVENTION

Injection devices which are used especially for freshly mixed bone cements differ from those for liquids in that the narrowest passage opening for the material to be injected is critical and that the ratio of the diameter of the narrowest passage opening to the diameter of the cavity of the syringe body should not be less than a particular minimum value. If the ratio falls below such a minimum value, excessively high injection pressures result when the injection device is used in surgery, particularly at the spinal column. High injection forces require the surgeon to apply a greater force and the injection device, which consists of plastic, to be more resistant to mechanical breakage. At very high injection forces, it is possible that the surgeon is no longer in a position to inject the cement or that the plastic syringe could break. There is therefore a need for injection devices, which can be operated with a lower injection force.

In spinal column surgery, it is extremely important to use a highly viscous bone cement, because this reduces the danger that the bone cement can flow out into the spinal canal. However, a highly viscous bone cement leads to high injection forces. Accordingly, the need for injection devices with a lower injection force is even more important for surgery of the spinal column, because the risks associated with this technique can be reduced with such a device.

A syringe, which can be connected with a cannula by means of a luer lock, is known, for example, from U.S. Pat. No. 4,220,151. This known luer lock connection is distinguished owing to the fact that two sleeves, which are disposed concentrically, one within the other, are mounted at the front end of the syringe. The outer sleeve is provided with an internal thread, into which the rear end of the cannula can be screwed. On the outside, the inner sleeve has a cone, which forms an air-tight conical connection with the complementary, conically constructed central borehole at the rear end of the cannula when the latter is screwed in. It is a disadvantage of this known device that the maximum diameter of the central borehole, at the transition between the syringe and the cannula, is limited appreciably by this inner sleeve.

U.S. Pat. No. 4,993,948 (CAMERON et al) discloses an injection device for dental filling materials, which has a transition segment with a borehole, which, at the transition between the cavity of the syringe body and the borehole of the connecting piece, has a cross-section, which is larger than the lumen of the cannula at the rear end of the latter.

U.S. Pat. No. 4,338,925 (MILLER) discloses a further injection device for bone cement, which comprises a cannula of constant cross section.

Finally, WO 01/52924 (ULTRADENT PRODUCTS) discloses a syringe, which is equipped with a luer lock connection and suitable for mixing and administering a viscous composition.

### BRIEF SUMMARY OF THE INVENTION

It is an object of the invention to provide an injection device, which comprises a connecting piece for a cannula, the narrowest passage opening for the material, which is to be injected, being as large as possible at the transition between the connecting piece and the cannula and not falling below a critical value.

Pursuant to the invention, this objective is accomplished with an injection device.

The advantages, achieved by the invention, can be seen to lie essentially therein that, due to the inventive injection device,
a diameter for the outlet opening and for the central borehole of the cannula, which is large relative to the diameter of the cavity of the syringe body, can be attained,
an appreciable reduction in the injection forces can be achieved
in comparison to the state of the art, a lower pressure is possible, and
it is possible to use the device for bone cements in the region of the spinal column.

In a preferred embodiment, the central borehole of the cannula has a constant cross-sectional area q in the axial direction. The advantage of this embodiment lies therein that the injection forces are not increased by constrictions.

In a different embodiment, the cavity of the syringe body has a cross-sectional area Q≦q, which is orthogonal to the longitudinal axis, the ratio of the cross-sectional area q to the cross-sectional area Q being between 1 and 0.01 and, preferably, between 1 and 0.02.

In yet another embodiment, the cavity has a cross-sectional area Q>q, which is orthogonal to the longitudinal axis, the ratio of the cross-sectional area q to the cross-sectional area Q being between 0.200 and 0.033 and preferably between 0.2 and 0.05.

Due to the selection of a suitable value for the ratio of the cross-sectional areas Q and q, the connection between the syringe body and the cannula can be adapted optimally to the properties of the material to be injected, especially with regard to the injection forces. A large ratio of q:Q is selected for highly viscous bone cements.

In a further embodiment, the borehole in the connecting piece has an internal thread, which may be formed, for example, as a sawtooth thread. At its rear end, the cannula has means, by which it can be screwed into the internal thread, the means being constructed as cams or as a complementary external thread. By these means, the elements, connecting the body of the syringe and the cannula, can be produced easily.

In yet another embodiment, the connecting piece is constructed as a luer lock adapter without the internal conical element. With that, the advantage can be achieved that a conventional commercial cannula with a luer lock adapter can be connected with the connecting piece of the body of the syringe. Preferably, the diameter of the borehole in the transition segment between the cavity and the connecting piece and the geometry of the internal thread in the connecting piece correspond to the standardized dimensions of a luer lock connection. The means at the cannula for screwing into the internal thread may be constructed completely as a luer lock adapter.

### BRIEF DESCRIPTION OF THE DRAWINGS

The invention and further developments of the invention are explained in even greater detail in the following by means of the partially diagrammatic representation of several embodiments. In the drawings:

FIG. 1 shows a longitudinal section through an embodiment of the inventive injection device and

FIG. 2 shows a section along the line II-II of FIG. 1.

### DETAILED DESCRIPTION OF THE INVENTION

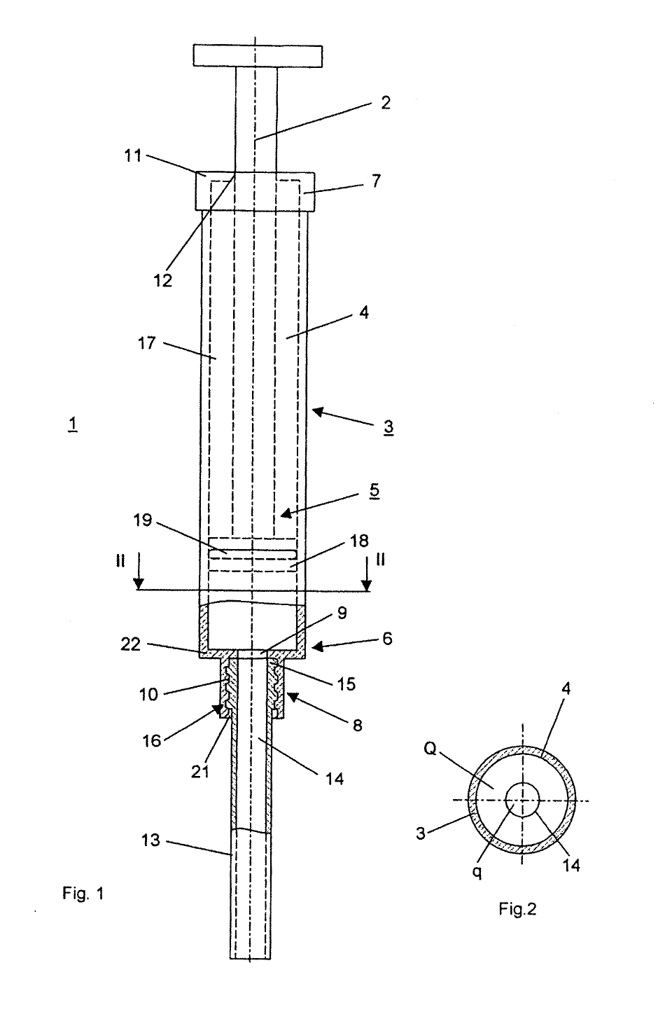

In FIGS. 1 and 2, an embodiment of the injection device 1 is shown, which comprises a syringe body 3, which is coaxial with a longitudinal axis 2, an injection plunger 5, which can be shifted axially in the cavity 4 of the syringe body 3, and a cannula 13, which can be fastened detachably to the front end 6 of the syringe body 3. The rear end 7 of the syringe body 3 can be closed off by means of a covering cap 11 having a coaxial, continuous opening 12. The diameter of the opening 12 is selected so that the piston rod 17 of the injection piston 5 is supported axially displaceably in the opening 12. Furthermore, the injection piston 5 comprises a piston 18, which is fastened at the front end 14 of the piston rod 13, and peripherally has a sealing element 19, by means of which the space between the piston 18 and the wall 20 of the cavity 4 is sealed. The sealing elements may consist, for example of Viton, polypropylene, polybutyl or Teflon.

At its front end 6, the syringe body 3 has a transition segment 27, which has a coaxial borehole 9 and ends in the connecting piece 8. The connecting piece 8 is provided with a borehole 21, which is coaxial with the longitudinal axis 2. The borehole 21 of the connecting piece 8 is provided with an internal thread 10. The rear end 15 of the cannula 13 is provided with means 16 for screwing the cannula 13 into the internal thread 10. In this case, the means 16 are configured as an external thread, which is complementary to the internal thread 10. The central borehole 14 of the cannula 13, which is coaxial with the longitudinal axis 2, is constructed as a circular cylinder with a diameter d and has a cross sectional area q orthogonal to the longitudinal axis 3, whereas the cavity 4 of the syringe body 3 has a cross sectional area Q>q, which is orthogonal to the longitudinal axis 3. Furthermore, the borehole 9, which is disposed coaxially at the transition segment 22 and connects the cavity 4 with the central borehole 14, also is circularly cylindrical with the same diameter d, so that the central borehole 14 at the rear end 15 of the cannula 13 is aligned with the borehole 9 in the transition segment 22 at the front end 6 of the syringe body 3.

1. An injection device (1) especially for bone cement, comprising:
A) a syringe body (3) with a longitudinal axis (2), a front end (6), a connecting piece (8), disposed at the front end (6) and having a coaxial borehole (21), and a coaxial cavity (4);
B) an injection piston (5), which can be shifted coaxially in the cavity (4); and
C) a cannula (13), which can be connected with the connecting piece (8), with a central borehole (14) and rear end (15); wherein
D) the front end (6) of the syringe body (3) has a transition segment (22) with a coaxial borehole (9) with constant diameter, connecting the cavity (4) with the borehole (21) in the connecting piece (8); and wherein
E) the borehole (9) in the transition segment (22) and the central borehole (14) have the same cross-sectional area orthogonal to the longitudinal axis (2) at least at the rear end (15) of the cannula (13).

2. The injection device (1) of claim 1, wherein the central borehole (14) of the cannula (13) has a constant cross-sectional area q in the axial direction.

3. The injection device (1) of claim 1, wherein the central borehole (14) of the cannula has a cross-sectional area q and wherein the cavity (4) has a cross-sectional area Q, which is orthogonal to the longitudinal axis (2), and that the ratio of the cross sectional areas q:Q is between 1 and 0.01 and preferably between 1 and 0.02.

4. The injection device (1) of claim 1 wherein the central borehole (14) of the cannula has a cross-sectional area q and wherein the cavity (4) has a cross-sectional area Q, which is orthogonal to the longitudinal axis (2) and that the ratio of the cross sectional areas q:Q is between 0.200 and 0.033 and preferably between 0.2 and 0.05.

5. The injection device (1) of claim 1, wherein the borehole (9) has an internal thread (10) in the connecting piece (8).

6. The injection device (1) of claim 5, wherein the cannula (13) at the rear end (15) comprises means (16) for screwing the cannula (13) into the internal thread (10).

7. The injection device (1) of claim 6, wherein the means (16) are an external thread, which is complementary to the internal thread (10).

8. The injection device (1) of claim 1, wherein the connecting piece (8) is constructed as a luer lock adapter without an internal conical element.

9. The injection device (1) of claim 5, wherein the diameter of the borehole (9) in the transition segment (22) and the geometry of the internal thread (10) in the connecting piece (8) correspond to those of a luer lock connection.

10. The injection device (1) of claim 6, wherein the means (16) for screwing into the internal thread (10) are constructed as a luer lock adapter.

11. The injection device (1) of claim 2, wherein the cavity (4) has a cross-sectional area Q, which is orthogonal to the longitudinal axis (2), and that the ratio of the cross sectional areas q:Q is between 1 and 0.01 and preferably between 1 and 0.02.

12. The injection device (1) of claim 2 wherein the cavity (4) has a cross-sectional area Q, which is orthogonal to the longitudinal axis (2) and that the ratio of the cross sectional areas q:Q is between 0.200 and 0.033 and preferably between 0.2 and 0.05.

13. The injection device (1) of claim 6, wherein the diameter of the borehole (9) in the transition segment (22) and the geometry of the internal thread (10) in the connecting piece (8) correspond to those of a luer lock connection.

14. The injection device (1) of claim 7, wherein the diameter of the borehole (9) in the transition segment (22) and the geometry of the internal thread (10) in the connecting piece (8) correspond to those of a luer lock connection.

15. The injection device (1) of claim 7, wherein the means (16) for screwing into the internal thread (10) are constructed as a luer lock adapter.

16. The injection device (1) of claim 9, wherein the means (16) for screwing into the internal thread (10) are constructed as a luer lock adapter.
